(19) United States
(12) Patent Application Publication
(10) Pub. No.: US 2009/0137910 A1
(43) Pub. Date: May 28, 2009

(54) ORGAN OXYGENATION STATE MONITOR AND METHOD

(76) Inventors: Jason L. Pyle, Redwood City, CA (US); Alexander M. Aravanis, San Diego, CA (US); Jeffrey N. Roe, San Ramon, CA (US); Myer H. Rosenthal, Stanford, CA (US)

Correspondence Address: Karen Drexler, 12580 La Cresta Dr, Los Altos Hills, CA, 94022, US

(21) Appl. No.: 11/946,055

(22) Filed: Nov. 28, 2007

(51) Int. Cl. A61B 5/00

(52) U.S. Cl. 600/478

(57) ABSTRACT

One aspect of the invention provides a patient tissue state monitoring system with a light source; a light detector; a probe adapted to be inserted into a patient to transmit light from the light source to an organ tissue site and to direct light from the organ tissue site to the detector; and a processor programmed to determine tissue state with respect to a tissue site pre-dysoxia point from a fluorescence emission detected by the detector (such as by determining tissue NADH concentration) and to provide an indication of tissue state through an output device (such as by displaying a numerical value corresponding to the fluorescence emission). Another aspect of the invention provides a method of monitoring a patient tissue state including the following steps: monitoring an aerobic energy production level of an organ tissue site (such as tissue within the patient's gastro-intestinal tract, bladder and/or urethra); determining tissue state with respect to a tissue site pre-dysoxia point from the monitored aerobic energy production level; and providing an output of the tissue state (such as by displaying a numerical value corresponding to the fluorescence emission).

### CROSS REFERENCE TO RELATED APPLICATIONS

This application is related to U.S. patent application Ser. No. 11/473,976, filed Jun. 23, 2006, which claims priority to U.S. Patent Application No. 60/595,337, filed Jun. 23, 2005, the disclosures of which are incorporated by reference as if fully set forth herein.

### BACKGROUND OF THE INVENTION

FIG. 1 shows a diagram of aerobic energy production to illustrate the inputs and outputs of this mitochondrial-based process. The inputs are the electron donors, NADH and FADH₂, and molecular oxygen O₂. The output is ATP, the main source of cellular energy used to fuel all cellular processes. In normal function, cellular energy is produced to match the demands of both cell sustenance, the minimum processes necessary to stave off cell death, and all of the higher-energy cellular functions that contribute to the function of the tissue, organ, and organism as a whole.

During glycolysis, a single molecule of glucose is converted into two molecules of glyceraldehyde-3-phosphate (G-3-P). The energy of the subsequent G-3-P oxidation reaction is conserved in the formation of NADH from NAD+. In the presence of oxygen, the conversion of pyruvate to acetyl-CoA yields two molecules of NADH. Three more molecules of NADH are formed for every turn of the citric acid cycle. The process of glucose metabolism in the presence of oxygen generates a total yield of ten NADH molecules for every molecule of glucose. The ten molecules of NADH are converted into thirty molecules of ATP as electrons pass from NADH to molecular oxygen through a chain of electron carriers. Thus, the stoichiometry of NADH/NAD+ is shifted heavily toward the production of NAD+ in the presence of oxygen, while the stoichiometry of NADH/NAD+ is shifted toward NADH in anaerobic conditions. Measurement of cellular NADH therefore reflects a direct measurement of the energy production status of a cell, a process intimately tied to the availability of molecular oxygen.

In the setting of the hospitalized patient, cellular energy production is most frequently compromised by inadequate oxygenation of end organ tissues. Even when cardiac output and measured oxygen saturation of hemoglobin are normal, end organ tissues may still not receive adequate oxygenation. This condition is especially worrisome during the inflammatory processes that accompany sepsis and septic shock, as well as in the presence of the many vasoactive substances used in anesthesia and critical care.

One method of determining tissue dysoxia is by measuring the redox state of cellular NAD in the cells of a tissue or organ, especially in a patient's vital organs. High cellular NADH relative to NAD+ is usually indicative of dysoxia. This is because NADH is converted to NAD+ during oxidative phosphorylation in the mitochondria. If oxidative phosphorylation decreases due to lack of oxygen, there is build up of NADH. Therefore, by monitoring the redox state of NAD in a patient's vital organs, dysoxia can be detected. Using this approach, when and if dysoxia occurs, the physician will be aware and can take the necessary medical steps to reverse or minimize the condition. The course of action usually involves the administration of various hemodynamic agents to increase the output of the heart and/or manipulate the vascular resistance. Alteration in blood pressure can occur either through changes in cardiac output or vascular resistance. Therefore, the management of patients with hypotension requires the clinician to determine which of these components needs manipulation. Both the selection of type of therapy and the determination of the patient's response to that therapy (magnitude and direction) require immediate assessment of the response in tissue oxygenation utilization, which is clinically unavailable at the present time.

Since NADH has intrinsic fluorescent properties, spectroscopic tissue monitoring and analysis systems based on these properties have been proposed. See, e.g., Renault U.S. Pat. No. 4,449,535. For example, U.S. Patent Application. Pub. No. 2005/0234315 describes a device that purports to monitor a patient's “metabolic emergency state” by detecting fluorescence of NADH and other “tissue viability.” The meaning of “metabolic emergency state” is not well defined, however, nor is there any indication of how that information would be used clinically.

### SUMMARY OF THE INVENTION

In critical care hospital settings such as the intensive care unit (ICU) and high-risk surgery, patients are at risk for tissue dysoxia and subsequent organ failure. Dysoxia occurs when an organ's tissue oxygen demand is greater than the oxygen supplied to the tissue. One consequence of inadequate oxygen delivery is a conversion from aerobic to anaerobic cell metabolism within the tissue yielding insufficient amounts of ATP and production of lactic acid. The resultant fall in tissue pH with the development of a metabolic acidosis further impedes vital organ perfusion and function and uncouples nearly all enzymatic chemical reactions between the ischemic tissue and the rest of the patient's body. Ischemia of the gastrointestinal (GI) tract is a major source of lactic acid production.

If dysoxia is severe and persistent the affected organs will become ischemic and eventually infarcted. Along with the damage to the organ tissue there is a concomitant decrease in organ function, and eventually complete organ failure may occur. It is well documented that patients having even a single failed organ have a dramatically higher mortality rate. For these reasons, it is of paramount importance to prevent organ failure. The key to preventing organ failure is to insure that organ dysoxia does not occur or is minimized. This leads to the need to not only detect the presence and severity of tissue dysoxia but also to warn of impending dysoxia.

Thus, knowledge of the level of aerobic energy production in a patient's tissue is valuable information that can be used to make critical health care decisions. In the clinical setting, restricted oxygen delivery to tissue becomes the rate-limiting component of aerobic energy production. As aerobic production of ATP decreases, higher-cellular functions are shut down. A further decrease in aerobic ATP production will eventually reach a level at which the minimum cellular processes can no longer be sustained, and cell death ensues. Three particularly important levels of ATP production are (1) the pre-dysoxia point, when aerobic energy production no longer has excess oxygen, (2) the dysoxia point, when aerobic metabolism actually becomes limited by the oxygen supply, and (3) the critical point, when aerobic production of ATP is less than the requirements for minimal sustenance of cellular processes.

The redox state of NAD in a patient can be measured in a minimally invasive manner using an optical probe attached to a light conduit. The light conduit is in turn attached to a spectroscopic instrument. The system functions by having the spectroscopic instrument send light to the tissue of interest via the optical conduit and probe. The light emitted from the tissue as a consequence of irradiation is then collected by the optical probe and then transmitted back to the spectroscopic device. By analyzing the emitted light, the redox state of NAD in the irradiated tissue can be determined and communicated to an attending healthcare provider.

The invention includes a method and a device for determining a level of aerobic energy production in a patient and its relation to the clinically important levels of pre-dysoxia point, dysoxia point and the critical point.

One aspect of the invention provides a patient tissue state monitoring system with a light source; a light detector; a probe adapted to be inserted into a patient to transmit light from the light source to an organ tissue site and to direct light from the organ tissue site to the detector; and a processor programmed to determine tissue state with respect to a tissue site pre-dysoxia point from a fluorescence emission detected by the detector (such as by determining tissue NADH concentration) and to provide an indication of tissue state through an output device (such as by displaying a numerical value corresponding to the fluorescence emission). In some embodiments, the patient tissue state monitoring system also includes a light conduit extending distally from the light source to the probe, and in some embodiments the patient tissue state monitoring system includes a light conduit extending proximally from the probe to the light detector. In some embodiments, the processor is further programmed to determine tissue state with respect to a tissue site dysoxic point and/or with respect to a tissue site oxygenation critical point from a fluorescence emission detected by the detector.

Another aspect of the invention provides a method of monitoring a patient tissue state including the following steps: monitoring an aerobic energy production level of an organ tissue site (such as tissue within the patient's gastro-intestinal tract, bladder and/or urethra); determining tissue state with respect to a tissue site pre-dysoxia point from the monitored aerobic energy production level; and providing an output of the tissue state (such as by displaying a numerical value corresponding to the fluorescence emission). In some embodiments, the monitoring step includes the step of monitoring fluorescence emission of the organ tissue site to, e.g., determine tissue NADH concentration. Some embodiments also include the step of determining tissue state with respect to a tissue site dysoxic point and/or tissue site oxygenation critical point from the monitored aerobic energy production level.

Yet another aspect of the invention provides a method of warning of a likely pre-dysoxia point of a kidney including the following steps: monitoring an aerobic energy production level of a stomach tissue site; determining a stomach tissue site tissue state with respect to a tissue pre-dysoxia point from the monitored aerobic energy production level; and providing an output that the stomach of the tissue state (such as by displaying a numerical value corresponding to the fluorescence emission). In some embodiments, the monitoring step includes the step of monitoring fluorescence emission of the organ tissue site to, e.g., determine tissue NADH concentration.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a diagram of aerobic energy production to illustrate the inputs and outputs of this mitochondrial-based process.

FIG. 2 is a flow chart showing a method according to one aspect of the invention.

FIG. 3 is a flow char showing a method according to another aspect of the invention.

FIG. 4 illustrates the relationship between mitochondrial NADH concentration, cellular oxygen utilization and aerobic production of ATP.

FIG. 5 shows use of the monitoring of mitochondrial NADH concentration and tissue oxygen uptake in the splanchnic organs to inform the clinical status of renal function and acute renal failure.

FIG. 6 shows changes in mitochondrial NADH concentration is response to a decrease in tissue oxygen uptake.

FIG. 7 shows a tissue monitoring system according to one aspect of the invention.

### DETAILED DESCRIPTION OF THE INVENTION

Some embodiments of the present invention provide methods and devices for measuring, in vivo, the relative or absolute concentration of endogenous fluorophores in a tissue site and for using that information to provide useful information regarding impending dysoxia of the patient's vital organs. Suitable tissue sites include but are not limited to oral mucosa, esophageal mucosa, gastric mucosa, intestinal mucosa, bladder mucosa, and the like.

For the purpose of this specification, the synonymous terms, “intrinsic emission molecule” and “endogenous fluorophore” both refer to a molecule naturally present in mammalian cells that emits light following absorption of electromagnetic energy (fluorescence or phosphorescence) or chemical action (luminescence). In general, the method of monitoring the energy production status of end organ tissues is performed by transmitting excitation light onto the end organ tissue and measuring the fluorescence of intrinsic emission molecules. The close relationship between the fluorescence of intrinsic emission molecules, such as NADH, and the cellular energy production status, has been well established in the scientific literature. By measuring the fluorescence emission of intrinsic emission molecules at the site of end organ tissues, such as the mucosa of the gastrointestinal tract, end organ energy production status can be monitored in a medically beneficial manner. Moreover, this also allows for the medically beneficial monitoring of in vivo tissue oxygenation since this parameter is closely related to the end organ energy production status. This information can also be used to predict the oxygenation state of other organs that have not been, and perhaps can not be, monitored.

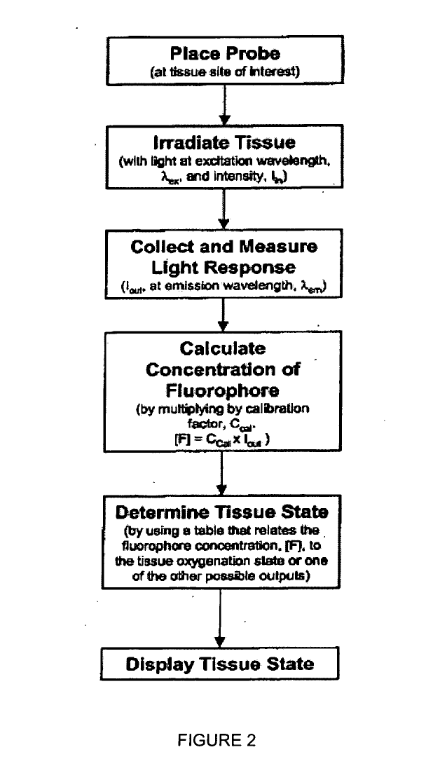

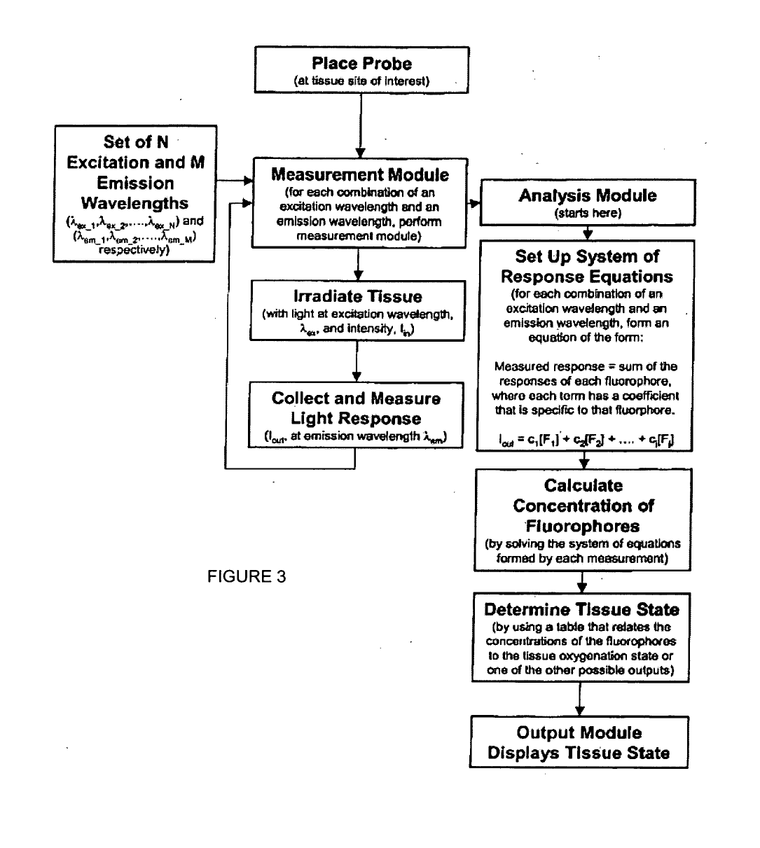

A known excitation wavelength is selected for the endogenous fluorophore in a range of wavelengths at which the endogenous fluorophore and/or chromophore undergoes fluorescence. The tissue site is irradiated with radiation having at least the selected excitation wavelength within the range. Fluorescence emission is detected of the tissue site resulting from the irradiation thereof. The detected emission is analyzed to determine the relative or absolute concentration of the endogenous fluorophore and/or chromophore in the tissue site. The measurements are used to estimate the in vivo cellular energy production status and state of end organ tissue oxygenation, as illustrated in the flow charts of FIGS. 2 and 3.

In one embodiment, following irradiation of the tissue site, the intensity of the emitted light is measured for a wavelength known to be in the emission spectra of this fluorophore. An estimate or calculation is then made of the relative or absolute concentration of the fluorophore or by multiplying the intensity by a calibration factor that depends on but is not limited to one or more of the following: the known excitation and emission properties of the fluorophore, the intensity of the irradiated light, the optical properties of the measurement system, and the specific properties of the tissue site being irradiated. From the estimate or calculation of the concentration of this fluorophore, estimations are then made for the important clinical parameters of in vivo cellular energy production status and state of end organ tissue oxygenation. This estimate is based either upon known relations between the concentration of this fluorophore and these clinical parameters or upon an experimentally derived calibration. This result is then displayed in one or more forms described hereafter.

In another embodiment, the tissue site is irradiated at multiple wavelengths known to excite one or more fluorophores interest. For each excitation wavelength, the intensity of the emitted light is measured at one or more wavelengths known to be in the emission spectra of one or more fluorophores. For each measurement, involving irradiation at a certain wavelength and a measured response at a certain emission wavelength, an equation is created in the form where the measured response is equal to the sum of the responses from all the fluorophores. The term representing the response of each fluorophore is proportional to the fluorophore concentration and one or more constants, some of which may represent the unique spectral properties of the particular fluorophore. It should be noted that the concentration of certain fluorophores are closely related to tissue oxygenation state whereas others do not vary with tissue oxygenation state and whose emission signals represent background signals.

A system of equations is formed, where there is an equation for each combination of irradiation wavelength and measured emission wavelength. This system of equations is solved for the absolute or relative concentrations of each of the fluorophores. In another embodiment, instead of solving the system of equations directly, the ratios of measurements at different excitation and/or emission wavelengths is performed to obtain the concentration of one or more fluorophores of interest. The value of doing measurements at multiple excitation and emission wavelengths is that it allows for correction of background fluorophores, instrumental variation, and positioning of the probe. From the calculation of the concentration of these one or more fluorophores, an estimation is then made of the in vivo cellular energy production status and state of end organ tissue oxygenation. This estimate is based either upon the known relations between the concentrations of these fluorophores and the in vivo cellular energy production status and state of end organ tissue oxygenation or upon an experimentally derived calibration. These results are then displayed.

A number of additional signals may be additionally captured and used to correct, calibrate, or augment some signal composed of an electron donor molecule such as NADH or FADH₂ in either its reduced or oxidized forms. In addition, a number of numerical forms may be used to adequately convey the clinical endpoints to practitioners; these include, but are not limited to absolute values, ratios, or other algebraic manipulations of reduced or oxidized molecules associated with mitochondrial energy production.

One practical use of this measurement comes from the identification of one or more clinically important levels of organ tissue oxygenation, such as the pre-dysoxia point (when tissue aerobic energy production no longer has excess oxygen), the dysoxia point (when aerobic production of ATP becomes limited by the tissue oxygen supply) and the critical point (when aerobic production of ATP is less than the requirements for minimal sustenance of cellular processes in the tissue). FIG. 4 illustrates the relationship between mitochondrial NADH concentration, cellular oxygen utilization and aerobic production of ATP. In this figure, VO_{2,max} is maximum cellular oxygen utilization, VO_{2,pre-dysox} is the cellular oxygen utilization at the transition between the regime where oxygen is in excess to the regime where although aerobic energy production is still maintained, it requires compensatory changes in stoichiometry and/or enzymatic reaction rates, VO_{2,dysox} is oxygen utilization at the dysoxic transition when aerobic energy production is actually limited by oxygen, VO_{2,crit} is oxygen utilization at the critical transition, NADHₙₒᵣₘ is NADH concentration in the region of oxygen excess, NADH_{pre-dysox} is NADH concentration at the transition between the regime of oxygen excess and the regime where the aerobic energy production is maintained through compensatory changes in stoichiometry and/or enzymatic reaction rates, NADH_{dysox} is NADH concentration at the dysoxic transition, NADH_{crit} is NADH concentration at the critical transition, and NADHₘₐₓ is maximum NADH concentration.

FIG. 4 shows four clinically significant regions of tissue oxygen uptake under the NADH concentration curve. R1 is the region in which the tissue has excess oxygen. In other words, energy production of ATP is at baseline and is not oxygen limited. ATP production is matched to demand, NADH concentration is low and stable, and organ function is normal.

R2 is the region of biochemical adaptation. As oxygen supply and uptake decreases, compensatory changes in redox and/or phosphorylation ratios maintain baseline energy production of ATP. Energy production of ATP is still not oxygen limited, and the cell is not dysoxic despite limited oxygen supply. ATP production is matched to demand, NADH concentration has increased, and organ function is normal.

R3 is the region of dysoxia in which aerobic production of ATP is limited by oxygen supply. Energy production of ATP is still near baseline due to an increase in anaerobic production of ATP. ATP production is matched to demand, NADH concentration has further increased, and organ function is potentially impaired.

R4 is the region of impaired cell function. ATP production by any means is now insufficient for the cell to perform its most vital functions. Compromise of cell integrity and cell death will eventually occur. ATP production no longer meets demand, NADH concentration has increased still further, and organ function is impaired perhaps to the point of organ failure.

As tissue oxygen supply is decreased, oxygen utilization by the tissue becomes limited. There is a commensurate increase in NADH concentration as oxygen utilization decreases. The point between R1 and R2 (VO_{2,pre-dysox}) is the point where oxygen is no longer in excess; changes in the biochemical pathway of aerobic ATP production occur to maintain energy for all required processes. The point between R2 and R3 (VO_{2,dysox}) is the point where aerobic production of ATP actually becomes limited by oxygen supply. VO_{2,crit}, the boundary between R3 and R4, is the point where oxygen supply is so low that the most essential life sustaining metabolic processes cannot be maintained. For each of these points there is a corresponding NADH concentration. This concentration can be measured and identified for a clinician using the instant invention.

There is also a relationship between NADH concentration and ATP production. The demand for ATP varies widely during normal physiologic function. The production of ATP is heavily dependent on the availability of ADP and therefore tightly matched to demand during normal cellular function. As a consequence, changes in ATP production that are driven by changes in demand are associated with little or no change in the concentration of NADH. Two points of ATP production are defined by the biochemical processes of metabolism: (1) the aerobic maximum, a state of ATP production where the metabolic machinery of the cell is functioning at its maximum possible rate; and, (2) the anaerobic state, where the cell is wholly reliant on ATP produced by glycolysis. Another point is defined, indicated on the graph as the “minimum sustenance”, a point below which ATP production is unable to support the basic needs of the tissue. At ATP production below this point, the tissue is in immediate danger of death. The point of minimum sustenance varies widely by tissue type. Some tissue can exist indefinitely at or near the anaerobic threshold. Other tissue, whose baseline energy requirements are much higher, have a point of minimum sustenance significantly greater than the anaerobic threshold.

The most important clinical state in patient management is the transition between regions R1 and R2 in FIG. 3, the “pre-dysoxic point.” Once tissues are at the pre-dysoxic point, further decrease in oxygen supply will result in organ malfunction and eventually organ failure.

Hypoperfusion of abdominal organs, such as the GI and renal system, is a commonplace occurrence in clinical management. When perfusion is restored to these high-energy tissues, organ function may return immediately. However, in many situations the restoration of perfusion, particularly if delayed, does not lead to the immediate return of organ function, if ever. Severe tissue hypoperfusion results in tissue death. It is of enormous clinical importance to determine the difference between a failed organ (i.e., an organ that contains tissue whose energy production is currently not sufficient to perform the high energy functions of the organ) and an organ with impending tissue death (an organ that contains tissue whose energy production is not sufficient to sustain life). Furthermore, it may be of clinical interest to maintain certain organ systems such as GI, renal, and liver in a state of limited organ compromise, but not in a state that risks tissue death. The recognition of this dysoxic but life-sustaining state is unattainable with current devices, methods, and protocols of care in the absence of a measurement device and method that reports this essential physiology to the clinical practitioner. Ultimately, the key to successful resuscitation is the early recognition of decreases in oxygen delivery and/or utilization before tissue hypoxia results in irreversible tissue damage.

One embodiment of the invention provides a system that can be a bedside instrument of electronic and optical components connected to a disposable catheter that is designed for insertion into an end organ of interest. The splanchnic perfusion bed, supplying arterial oxygenated blood to the GI tract, and its close relationship to the blood flow in other important end organs, such as the liver and kidney, is particularly useful for end organ oxygenation monitoring. Currently used catheters, placed either through the nose or mouth, into the gut represents one embodiment of a method for the placement of an end organ oxygenation monitor.

Direct measurement of splanchnic organ status is of significant value to the practicing physician provided that some or all of the clinically useful points of pre-dysoxia, dysoxia, and minimum sustenance can accurately reported. The clinical value of a measurement in the splanchnic bed is further extended to the degree that measurements of energy status in the splanchnic bed inform on the status of renal tissue in a predictable manner. During the common clinical occurrence of abdominal organ hypoperfusion, renal perfusion (and therefore oxygen availability) will remain greater than splanchnic perfusion over a large number of pathologic conditions. This allows the anticipation of renal function and renal failure by direct measurement of the energy status of the splanchnic bed. As acute renal failure remains one of the most common and severe morbidities in hospitalized patients, and instrument that provided physicians with anticipatory knowledge of this event is of tremendous clinical value.

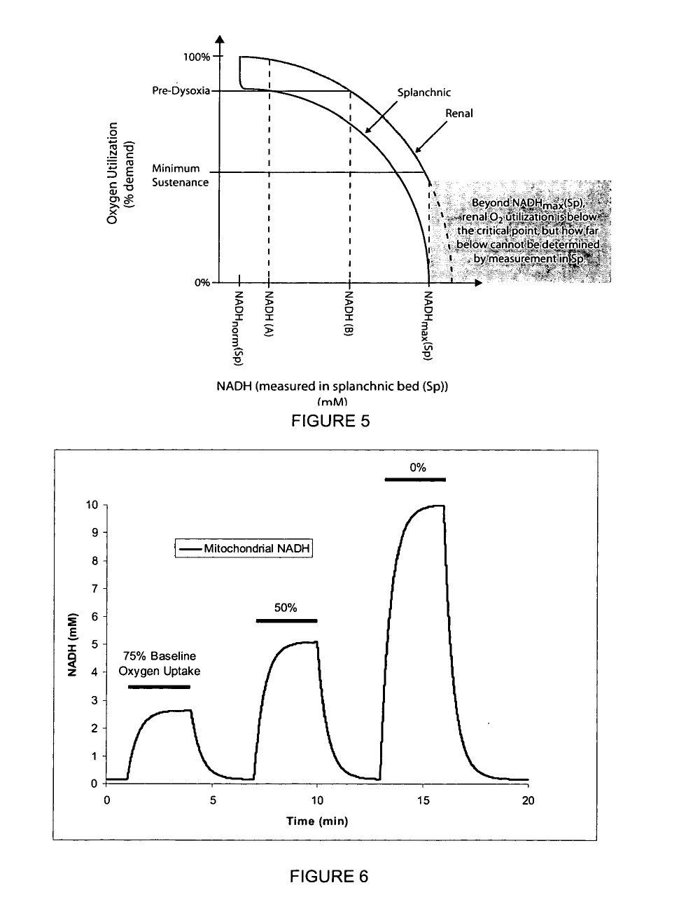

FIG. 5 shows use of the monitoring of mitochondrial NADH concentration and tissue oxygen uptake in the splanchnic organs to inform the clinical status of renal function and acute renal failure. The splanchnic organs are more sensitive to decreases in oxygen supply and uptake than the kidneys are. Therefore, during a patient perfusion crisis NADH concentration increases in the splanchnic organs before NADH concentration increases in the kidneys, thereby providing an early and sensitive indicator of insufficient oxygen supply and utilization.

Consider the clinically important point of pre-dysoxia. A measurement of NADH concentration in the splanchnic bed indicating pre-dysoxia is shown as NADH(A) on the graph of FIG. 5. In this example, while the splanchnic bed is pre-dysoxic, the renal tissue remains above the pre-dysoxic point. Only when the measurement of NADH in the splanchnic bed falls to the level indicated as NADH(B) has the renal tissue reached the pre-dysoxic point. In this example, both of these points are greater than the previously described minimum sustenance level, as shown on the graph. Thus, a patient tissue state monitor providing information that the pre-dysoxia point had been reached in, e.g., the stomach would provide a warning of an upcoming pre-dysoxia point in the kidneys. Note also that the point of NADHₘₐₓ(SP) indicates the maximum level of NADH measurable in the splanchnic bed. At this point, renal oxygenation is below the critical point.

FIG. 6 shows changes in mitochondrial NADH concentration is response to a decrease in tissue oxygen uptake. Note that even small changes in oxygen uptake cause large increases in the NADH signal.

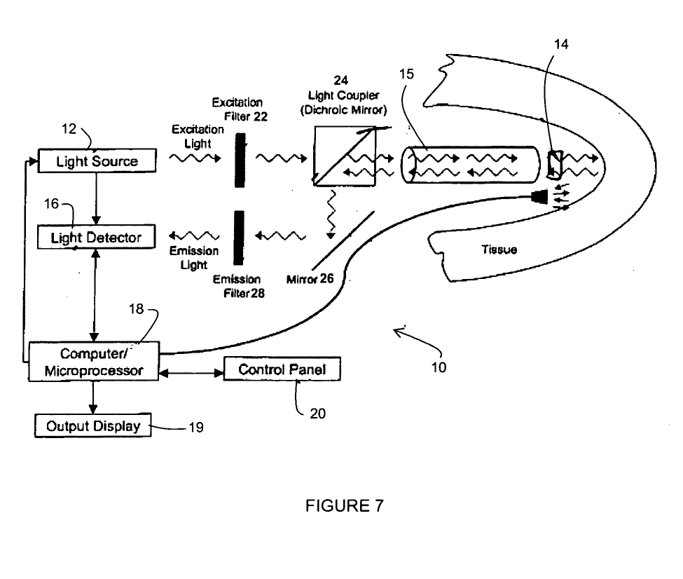

FIG. 7 shows a tissue state monitoring system 10 according to one embodiment of the invention for measuring in vivo the endogenous fluorophores in the tissue site and for providing an indication of tissue oxygenation state via a display or other output device. A light source 12 produces light at a wavelength in the excitation spectra of one or more of the endogenous fluorophores. A probe 14 suitable for placement at the tissue site of interest may be coupled to the light source with a coupler such as a light conduit 15 to irradiate the tissue with excitation light. The same probe 14 then collects the light emanating from the tissue site and transmits it to a detector 16 via the light conduit. The detector 16 measures this light, which is composed of emitted light from the fluorophores and possibly some of the excitation light that was incident on the irradiation site that has been scattered and reflected back into the probe. Suitable detectors include but are not limited to a photodiode, avalanche photodiode, charge-coupled devices (CCDs), photo-multiplier tubes (PMTs) and the like. Some embodiments might require multiple light sources if one source cannot provide all the required excitation wavelengths. Some embodiments might have multiple detectors which would allow simultaneous measurements at different wavelengths, rather than doing them sequentially. In alternative embodiments, the light source and detector may be located within the probe.

Resources, including but not limited to a processor 18, analyze the detected emission to determine the presence of the endogenous fluorophore in the tissue site. The processor 18 has sufficient memory and processing power to control the light source and detector, store the measured values of emission light, calculate the fluorophore concentrations using the analysis algorithm, determine the tissue state based on the fluorophore concentrations, and display this to an output device 19 such as an LED panel, LCD screen, or CRT screen.

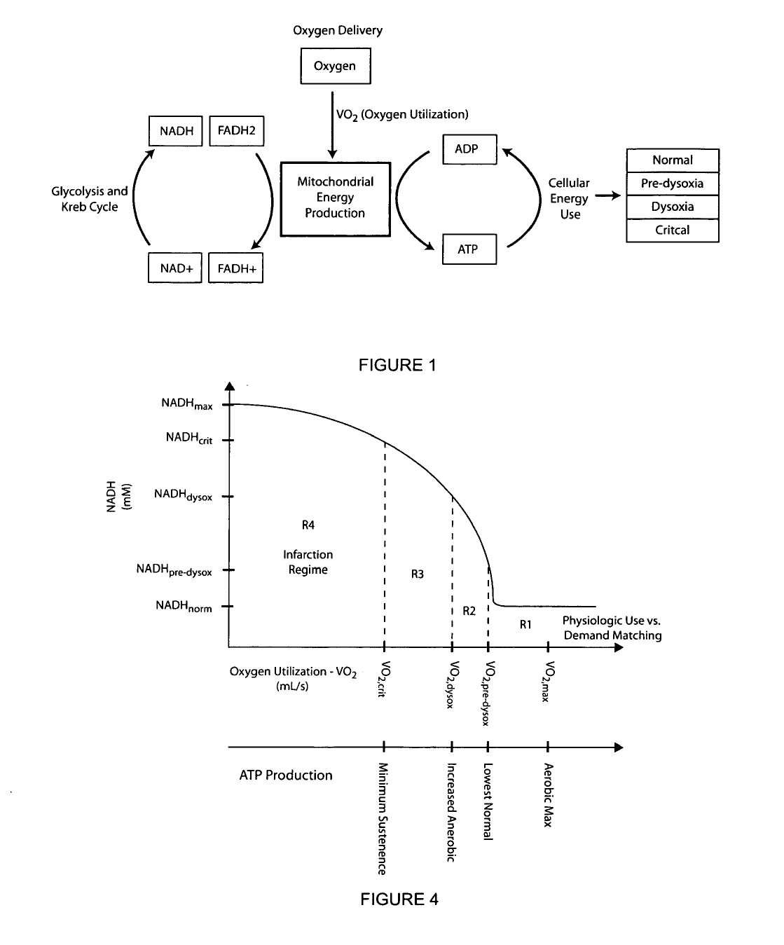

For all combinations of specified excitation wavelengths λₑₓ(1,2, . . . Nₑₓ) and emission wavelengths λₑₘ(1,2, . . . Nₑₘ) the processor 18 executes the information as shown in FIGS. 1 and 2.

For each excitation and emission wavelength pair, the sample response is represented as a sum of the responses from each chemical specie, and the processor 18 can execute the following algorithm:

1. D, a constant factor representing illumination intensity and wavelength independent effects on the optical pathway

2. λₑₓ(i), the ith excitation wavelength

3. λₑₘ(k), the kth emission wavelength

4. Cⱼ, the concentration of the jth chemical species

5. N_{c}, the number of chemical species

6. Tj(λₑₓ(i)λₑₘ(k)), the energy transfer function of the jth chemical species when illuminated at ith excitation wavelength and measured at the kth emission wavelength

7. R(λₑₓ(i),λₑₘ(k)), the measured sample response when it is illuminated at ith excitation wavelength and the response is detected at the kth emission wavelength.

The system of equations formed by all pairs is then solved for the concentration of each chemical specie.

$$R ( {λ}_{ex} ( i ) , {λ}_{em} ( k ) ) = D {\sum }_{j = 1}^{{N}_{c}} {C}_{j} {T}_{j} ( {λ}_{ex} ( i ) , {λ}_{em} ( k ) )$$

The light source can be any of the following, including but not limited to a diode laser, light-emitting diode (LED), metal halide lamp, gas arc lamp using xenon, mercury, or a halogen and the like. When the light source 12 is a laser, the laser can be a scanning laser for photon confocal imaging, a scanning laser for two-photon imaging, and the like.

In one embodiment, a measurement is made at one spatial location that corresponds to where the probe is placed in the body. Multiple measurements can also be made over a small region/patch of the tissue site. These measurements can be both parallel (along the organ or mucosal surface) and perpendicular (into the tissue). In one embodiment, when the light source is a scanning laser, two and three dimensional spatial measurements can be taken at surfaces parallel and perpendicular to a tissue site surface. Multiple measurements over a region of the tissue site permit a calculation of the tissue oxygenation gradient, which may be clinically useful information.

By way of illustration, and without limitation, suitable methods for making multiple two-dimensional, along the surface of the organ or mucosa, measurements include but are not limited to: (1) using an imaging endoscope to deliver and collect light; (2) if the light source is a scanning laser, scanning or moving the excitation beam across the tissue surface; or (3) performing simultaneous or sequential measurements through a bundle of optical fibers.

For measurements perpendicular to the tissue surface (going deep into the tissue) suitable methods include but are not limited to using: (1) confocal imaging (with a pinhole near the detector 16 to reject light that does not come from the deeper tissue plane of interest); (2) two photon imaging, i.e., focusing light that has a wavelength twice that of the desired excitation wavelength at the deeper tissue plane, so that excitation will only become effective close to the focus which is deep in the tissue; (3) using a graded-index (GRIN) lens that provides for a focusing of light coming out of a fiber or endoscope, and also allows more efficiently collect light emitted from the tissue, and the like.

In some embodiments, the probe 14 is a light delivery device that is coupled to the light source 12 and focuses the light from the light source 12 into the end of the probe 14. Suitable probes 14 include but are not limited to optical fibers that can be directly or indirectly coupled to the light source, a liquid-light guide, a catheter based probe, an endoscope and the like.

Probe 14 can be implantable. Suitable implantable probes 14 include but are not limited to, cochlear implants, pace makers, nerve stimulators, deep brain stimulators, and the like. The probe 14 can also be a partially implantable probe. Suitable partially implantable probes 14 include but not limited to, diabetic pumps, drainage tubes, implantable feeding tubes such as tubes connecting the intestines to an external port on the abdomen, and the like. The implantable probe 14 can have wireless control for calibration, changing mode of operation, real-time data output, data storage, data-retrieval, battery recharging, and the like. The implantable probe 14 need not need be wireless. In various embodiments, wires or tubes going into the body can be utilized and coupled to the probe 14 that can be surgically removed or simply pulled out at a time after the probe 14 is removed.

The probe 14 can be implanted in a variety of sites that include but are not limited to, the mucosal surface of certain organs such as the gastrointestinal tract and bladder, the parenchyma of other organs such as the kidneys, liver, lungs, heart, and the like. This can be especially useful during transplant surgeries where it is critical to monitor tissue oxygenation after you close incision.

In one embodiment, the system 10, light source 12, detector 16, processor 18 and the like, and not just the probe 14, is contained in a form that can be swallowed. Probe 14 can be a pill version that can be swallowed. In one specific embodiment, the probe 14 provides intermittent or continuous measurements while passing through the GI tract. The pill may also be retrieved after it is passed, allowing for subsequent retrieval of stored measurement data.

In various embodiments, the system calculates the absolute or relative concentrations of one or more molecules selected from the group: elastin, collagen, flavin adenine dinucleotide (FADH₂ and FAD²⁺, nicotinamide adenine dinucleotide (NAD(P)H and NAD(P)⁺), phenylalanine, pyridoxal 5′ phosphate, tryptophan, tyrosine and the like.

There are clinical scenarios where tissue can become deprived of oxygen, i.e., hypoxic or anoxic, include but are not limited to, embolic or thrombotic blood vessel stenois or occlusion as in but not limited to, (1) myocardial ischemia, infarction, and stroke of the brain, (2) organ transplantation, (3) shock of all types (septic, cardiogenic, hypovolemic) or (4) any other type of organ failure. These situations often arise in the acute care setting such as in an ICU or surgical suite. In all of these situations where there can be a decrease in tissue oxygen perfusion there will be also be changes in the redox state of NAD(P), FAD, and other fluorophores that can be measured. Initially, NAD(P) and FAD shift toward their reduced forms but eventually can switch towards the oxidized state as the tissue dies. The absolute or relative measurements of these fluorophores is then be used to estimate the oxygen perfusion state of the tissue.

In one embodiment, the processor 18 takes ratios of the measurements at different excitation and emission wavelength pairs to cancel out an effect of at least one of the following: distortions and variation caused by probe placement, optical and instrument distortions, and background signals. In one embodiment the processor uses measurements taken of at least one of selected excitation and emission wavelengths where selected species have near identical absorption or emission spectra to estimate an absolute or relative concentration of the sum of the selected species. In another embodiment, the processor uses measurements made at multiple spatial locations of the tissue site to determine spatial gradients of at least one of, chemical species, which is then used to determine spatial gradients in in vivo cellular energy production status and state of end organ tissue oxygenation.

These measurements can be used to determine quantities at the tissue site of at least one of, a percent of reduced NAD(P), a percent of oxidized NAD(P), a NAD(P)+/NAD(P)H ratio, a percent of reduced FAD, a percent of oxidized FAD, a FAD+/FADH₂ ratio, a percent of ischemia, a percent of perfusion, a percent of a perfusion deficit, a tissue state that is aerobic or anaerobic, and the like.

The system 10 can include a control module 20 to allow a user to change output displays or view data in a graphical form. The system can also include a GRIN lens, an excitation filter 22, light coupler such as a dichrotic mirror 24, mirror 26, emission filter 28 and the like.

In one embodiment, the probe 14 is coupled to an endoscope. In this embodiment, the endoscope functions as both the light conduit and the probe. In another embodiment, the probe 14 is coupled with a pulse oximetry system 30 to make measurements of tissue oxygenation verses blood oxygenation at the same tissue site.

The system 10 can include a mechanical device to anchor the probe 14 to tissue. A vacuum source, hooks, inflatable balloons, expandable cages, and non-toxic chemical adhesives can all be used to anchor the probe 14.

The following examples are possible clinical scenarios illustrating the organ oxygenation state method and monitor of this invention.

### EXAMPLE 1

In the case of shock of any kind, but especially septic shock, there is often multiple organ failure occurring secondary to decreased perfusion of the end organs with oxygenated blood. In this scenario, the probe is inserted into a hollow organ, such as the bladder, stomach or rectum, so the end organ tissue perfusion status is monitored.

In the case where monitoring from the rectum is desired, the probe will be sufficiently small (e.g., <1 cm in diameter) so as to easily pass through the anal sphincter. The probe may have light gathering and delivery features such as a GRIN lens attached to its distal end. The probe may also have mechanical and/or chemical adhesive features that promote its attachment and stable interface with intestinal mucosa. For example, the probe may use vacuum suction to attach to the intestinal mucosa. Alternatively, it may bend so as to wedge or lodge itself near the intestinal mucosa. The probe may also be attached to a light conduit such as a bundle of one or more optical fibers for the purpose of transmitting light from the light source to the probe and in turn transmitting light collected by the probe to the detector.

In operation, the clinician first places the patient in a position amenable for insertion, such as the lateral decubitus. The clinician then inserts the probe through the anal sphincter and advances the probe to a length consistent with desired site of monitoring. In the case of the rectum, this is somewhere between 0-15 cm.

The clinician then activates the system to measure the end organ oxygen perfusion in the rectum. The system takes its measurement by irradiating the intestinal mucosal surface with light at 380 nm and the emission response is measured at 410 nm and 470 nm. The signal at 410 nm represents mostly background signal that does not change with acute ischemia, while the signal at 470 nm reflects the amount of reduced NAD(P). The intensity of the emitted light is measured separately at 410 nm and 470 nm. The relative or absolute concentration of the reduced NAD(P) is then calculated by dividing the measured intensity at 470 nm by the measured intensity at 410 nm and then multiplying it by a calibration factor that depends on the known excitation and emission properties of the reduced NAD(P). From this calculation of the concentration of the reduced NAD(P), estimate is made of the in vivo cellular energy production status and state of end organ tissue oxygenation. This estimate is based either upon known relations between the NAD(P)H concentration and in vivo cellular energy production status and state of end organ tissue oxygenation or upon an experimentally derived calibration. This information is then displayed and can be used by the clinician to manage patient care accordingly.

### EXAMPLE 2

In the case of solid organ transplantation, maintaining sufficient end organ oxygen perfusion to the transplanted organ is critical to the survival of the organ. Examples of relevant organ transplantations where monitoring of end organ oxygen perfusion is beneficial, include but are not limited to the kidney, liver, heart, lung, intestines, limbs, fingers, cornea, and skin.

In the specific case of a kidney transplantation the probe is small, (e.g., <1 cm) so as to not interfere with transplantation surgery or take up significant volume in the abdominal cavity. The probe attaches to the outer surface of the kidney either through a mechanical means such as a vacuum suction or hooks, or by a chemical adhesive. This attachment is easily reversible and the probe can be removed with minimal to no additional surgery after monitoring is no longer needed. The probe may also be attached to a light conduit, such as a bundle of one or more optical fibers, for the purpose of transmitting light from the light source to the probe and in turn transmitting light collected by the probe to the detector.

In operation the clinician attaches the probe to the organ either before, during, or immediately after the organ transplantation surgery. The clinician activates the system to measure the end organ oxygen perfusion of the organ. The system determines the state of end organ oxygen perfusion using a method similar to that described in the case of septic shock above. The probe is left in place after the surgery is completed so measurements of end organ oxygen perfusion continue in the post-operative period. The probe remains connected to the rest of the system via the light conduit that would pass through a small opening in the abdominal cavity. When monitoring is no longer desired, the probe is removed by retracting it via the light conduit.

### EXAMPLE 3

In many clinical scenarios, including the case of shock mentioned above, it is more desirable to monitor end organ oxygen perfusion using the bladder as opposed to a site in the GI tract or other place. In this case the probe is sufficiently small enough to pass through the urethra (e.g., <5 mm). The probe also has a mechanism to keep it anchored in the bladder. This might be an inflatable balloon near the probe tip similar to that used in a Foley catheter. The probe and light conduit can be integrated into a Foley catheter system for simultaneous use. The probe tip is constructed in a manner such that when the anchoring system is deployed, the tip is placed in contact with the bladder mucosal surface. For example, the probe can be attached to the inflatable balloon in such a way that when it inflates the probe tip is pressed into the bladder mucosal surface. The probe is attached to a light conduit, such as a bundle of one or more optical fibers, for the purpose of transmitting light from the light source to the probe and in turn transmitting light collected by the probe to the detector.

In operation, the clinician lubricates the probe, inserts the probe through the urethral meatus and advances it until the probe enters the bladder. This distance is approximately 5 cm in women and 15 cm in men. The clinician activates the anchoring mechanism, which may be the inflation of a balloon near the tip of the probe. The clinician activates the system to measure the end organ oxygen perfusion in the bladder. The system determines the state of end organ oxygen perfusion using a method similar to that described in the case of septic shock above. When monitoring is no longer desired, the probe is removed by deflating the balloon and is retracted via the light conduit.

### EXAMPLE 4

The choice of pharmacologic agents to support blood pressure in septic shock is complex and often difficult to determine. In particular, the pharmacologic action of selected agents and the choice of amount and duration of these therapies are difficult to establish without the ability to assess the health of critical abdominal tissues (intestines, liver, and kidneys). Failure to recognize when therapy has led to end organ compromise leads to complications and death in patients, as illustrated in this case. Similar to the septic shock case described here, the same difficult choices regarding the correct therapy is common in patients with end-stage liver disease (i.e., cirrhosis). Patients with end-stage liver disease must often be managed in the context of hypotension and decreasing kidney function. Outcomes in these patients are questionable without better methods to direct therapy.

In this example, a 63-year-old man is brought to the hospital because he has become lethargic with a high fever and burning on urination. His evaluation reveals that he has a bacterial infection in his kidneys; he is placed on antibiotics. Over the next 6 hours in the hospital, he becomes hypotensive and obtunded. He is emergently brought to the intensive care unit where he is intubated and placed on a ventilator. He is noted to have a temperature of 39° C., a blood pressure of 100/45 mmHg and a pulse of 115 per minute. His peripheral arterial blood oxygenation is 100%. Arterial and central venous (CVP) catheters are placed and a diagnosis of septic shock is made. Fluids are administered to a normal CVP but he remains hypotensive.

A tissue state monitor according to this invention is placed in the upper GI tract. It is assumed that the reading of the monitor is completely reliable and precise indicator of both kidney and GI micro-perfusion. The output of the monitor is from 0-100. A measure of 100 represents fully healthy organs receiving excess oxygen and glucose. Measures of energy between 70 and 99 represent organs receiving enough oxygen and glucose to perform tissue function (organ compromise), with 70 representing the pre-dysoxia point for that tissue. Measures of energy between 50 and 69 represent organs receiving enough oxygen and glucose to survive indefinitely but not perform their tissue function (organ failure). Measures of energy between 20 and 49 represent organs not receiving enough oxygen and glucose to survive indefinitely. Measures of energy below 20 represent dead or imminently dying organs.

In addition to the stated vital signs, the monitor initially reads 90, suggesting that despite being hypotensive; the patient is receiving adequate (though not exceptional) perfusion to his abdominal organs. There are reasons beyond abdominal organ perfusion, including the adequacy of coronary oxygen delivery to the myocardium, that may lead the clinician to correct the patient's hypotension at this point. In addition to the perfusion of visceral end organs, it is imperative to maintain adequate perfusion to the heart and brain. Perfusion of the heart is largely a function of diastolic blood pressure (the only organ of the body for which this is true). Adequate perfusion and performance of the heart is routinely monitored and evaluated in operative and acute care settings by ECG. However, these measures are often inadequate. For example, the initial changes using ECG monitoring may be life threatening arrhythmias. Perfusion of the brain in a previously normotensive patient requires a mean arterial pressure of approximately 50 mmHg. Patients with cerebral injury or cerebral vascular disease may require much higher perfusion pressures.

### EXAMPLE 5

The facts are the same as in Example 4, except that the tissue state monitor initially reads 75, suggesting that while the pre-dysoxia state has not yet been reached, perfusion to the patient's abdominal organs is nonetheless considerably compromised. A clinical decision, following standardized protocols, is made to administer a vasoconstrictive drug. While standardized protocols may be of benefit in a number of complex clinical states to compensate for varying degrees of training, knowledge and experience of health care professionals, individualized adjustments of therapy based on careful precise assessments is an imperative necessity to optimize outcome and if available must be included in these protocols. Over the next 4 hours, the tissue state monitor falls to 60, indicating that tissue energy has fallen below that of productive organ function, though the tissue may still survive indefinitely. While the developing picture is concerning, the situation is not yet emergent. Nonetheless, therapy with the vasoconstrictive agent is not having the desired effect and if continued will likely result in dangerous vital organ ischemia. Addition of low-dose cardiac inotropic drugs and further fluids may be required at this time. The vasoconstrictive agent may need to be titrated down in order to be maximally effective.

### EXAMPLE 6

The facts are the same as in Example 5, except that the tissue state monitor reading has fallen to 20, indicating immediate organ failure and tissue death. Immediate resuscitation is required in this patient to avoid end organ failure. A complete reversal in strategy may be warranted for some period of time. The removal of vasoconstrictive agents and introduction of cardiac inotropes with immediate fluid bolus should be considered at this time.

### EXAMPLE 7

The facts are the same as in Example 4, except that the tissue state monitor initially reads 35, suggesting that the patient is already experiencing profound perfusion failure to the abdominal organs and impending organ death. It may be desirable for the clinician to administer a vasoconstrictive drug in this setting. Even though the vasoconstrictive drug may result in vasoconstriction of vasculature leading to the abdomen, the net effect of centralizing peripheral fluid may improve overall perfusion to the end organs. Nonetheless, this patient is in very serious condition with an extremely high chance for death. Immediate resuscitation of his end organ perfusion is required.

In the face of already failing end organs, it may also be reasonable to resuscitate this patient with cardiac inotropes before or in conjunction with vasoconstrictive drugs. While the long-term cardiac health is of concern in a patient on stringent inotropic therapy, the additional risk of morbidity and mortality posed by end organ failure outweighs longer-term considerations at this time. Immediate resuscitation of his end organ perfusion is required while it is still reversible. Longer-term management of his shock may revert to standardized protocols.

### EXAMPLE 8

When patients arrive at the hospital following a traumatic injury, it is often difficult to determine the volume of blood loss that has been sustained. Injuries such as a fractured leg, hip, or pelvis can generate profound internal bleeding. Rupture of internal organs, such as the spleen or liver, can bleed unrestricted into the abdomen. The goal of treatment is to quickly achieve evidence that the oxygen delivery to tissue is improving with continued resuscitation.

When patients are supported on oxygen ventilation in conjunction with cardiac inotropes and vasoconstrictors, the vital signs of arterial oxygenation, heart rate and blood pressure are not sufficient indicators of end organ perfusion. In this setting, it is difficult to assess whether end organ perfusion is improving or worsening. Mortality is unlikely in a young, previously healthy patient receiving this level of care by experience physicians; however, this patient's risk for acute renal failure and other morbid conditions is extremely high.

The skilled physician recognizes that tissue oxygenation is sustained by both the perfusion and the oxygen carrying capacity of blood. A common medical question in treating patients with blood loss is: when is the right time to administer blood? This situation presents itself both in trauma and in surgical patients. While some guidelines exist to determine when and how much blood is required, these therapies are not endpoint directed. In a patient whose cardiovascular system is already compromised, it is difficult to determine the necessary oxygen carrying capacity required for adequate tissue oxygenation.

In this example, a 34-year-old woman is brought to the emergency room following a motor vehicle accident in which she was the restrained driver of a car that ran off the road hitting a bridge abutment. In the field, she was noted to be conscious but with a low blood pressure and rapid pulse. Intravenous fluids were begun; she was brought to the emergency room. In the ER, her blood pressure is 60/40 mmHg and heart rate is 132 beats per minute. She is breathing rapidly but her peripheral arterial blood oxygen saturation is 100%.

A tissue state monitor is placed in her bladder along with a Foley catheter. It is assumed that the reading of the monitor is completely reliable and precise indicator of abdominal organ perfusion. The output of the monitor is from 0-100. A measure of 100 represents fully healthy organs receiving excess oxygen and glucose. Measures of energy between 70 and 99 represent organs receiving enough oxygen and glucose to perform tissue function (organ compromise) with 70 representing the pre-dysoxia point for that tissue. Measures of energy between 50 and 69 represent organs receiving enough oxygen and glucose to survive indefinitely but not perform their tissue function (organ failure). Measures of energy between 20 and 49 represent organs not receiving enough oxygen and glucose to survive indefinitely. Measures of energy below 20 represent dead or imminently dying organs.

In addition to the vital signs states, the initial reading of the tissue state monitor is 99, indicating full perfusion of the abdominal organs. However, additional intervention is warranted at this time: The patient's history of serious trauma combined with her vital signs is a worrisome picture. While it is reassuring that she is not currently experience end organ difficulties, this patient is unstable regardless of the current status of her end organ perfusion.

### EXAMPLE 9

The facts are the same as in Example 8. A second intravenous catheter is placed and normal saline is rapidly infused. Evaluation reveals a fracture of her left femur and evidence of hemorrhage in her abdomen. Blood is emergently ordered, and she is taken to the operating room for exploration of her abdomen. In the operating room, an arterial catheter is placed, and anesthesia is induced. Her blood pressure is now 90/50 mmHg, being maintained with vasoconstrictor agents intravenously. Fluid and O-negative blood are administered. The tissue state monitor reads 45, suggesting that the patient is experiencing profound perfusion failure to the abdominal organs.

This patient remains in serious and unstable condition despite the life-saving resuscitation she received in the emergency room. A blood pressure of 90/50 with 100% arterial blood oxygen saturation is providing adequate oxygenation to her heart and brain; however, she is currently experiencing abdominal end organ failure that will likely progress to serious complications if it is not corrected. Further measures need to be taken to restore her abdominal perfusion.

### EXAMPLE 10

The facts are the same as in Example 8. A ruptured spleen is found during laparotomy and is removed. She continues with low blood pressure, no urine output and a slight fall in peripheral oxygenation percentage. More intravenous fluids are administered and a central venous catheter is placed. The central venous catheter shows a low normal value of 6 mmHg. With light drug-induced vasoconstriction and fluids, her blood pressure is 100/60 mmHg and her pulse is 110 beats per minute. She continues without urine output. The tissue state monitor now reads 65 and is rising steadily.

This patient has been largely resuscitated. The source of internal bleeding has been removed and her vital signs are returning to normal. The tissue energy catheter indicates that abdominal perfusion has been restored and organ function has begun to normalize. The expectation at this time would be that her vital signs would stabilize and the tissue energy catheter would gradually move to 100.

### EXAMPLE 11

The facts are the same as in Example 8. A ruptured spleen is found during laparotomy and is removed. She continues with low blood pressure, no urine output and a slight fall in peripheral oxygenation percentage. More intravenous fluids are administered. With light drug-induced vasoconstriction and fluids, her blood pressure is 100/60 mmHg and her pulse is 110 beats per minute. She continues without urine output. The tissue state monitor now reads 40 and is not improving. Despite reasonable vital signs and the removal of her spleen, this patient has not been adequately resuscitated. Two possibilities remain: (1) the patient has adequate oxygen carrying capacity in her blood and is still not providing the abdominal organs with sufficient volume, or (2) an otherwise adequate perfusion of blood to the abdominal organs is not carrying enough oxygen to sustain them. Pulse oximetry, which only reads blood saturation independent of carrying capacity, does not shed light on this problem. We are concerned about the latter case, since much of this patient's blood volume has been replaced by normal saline. Further evaluation of this patient may reveal that her hematocrit is extremely low, a condition especially concerning in young women who are predisposed to normally low hematocrit levels.

### EXAMPLE 12

5% of all hospitalized patients develop acute renal failure; 60 to 70% of the cases are caused by prerenal hypoperfusion. A common operating room decision is whether or not to administer fluids in the face of poor urine output. Unfortunately, the skilled anesthesiologist recognizes that the low urine output may be due to a number of causes: inadequate fluid administration, effects of his vascular disease, or the expected increase in stress hormones as part of major surgery. The last condition routinely decreases urine output, but does not threaten kidney function. The administration of too much fluid leads to pulmonary edema and difficulty in oxygenation. The administration of too little fluid threatens the health of end organ tissue due to poor perfusion.

This case illustrates the use of diuretics to treat pulmonary edema. The treatment is routine but the end-point is often difficult to determine. If diuretics are used indiscriminately or without proper monitoring to determine the end-point for their use, renal failure may occur due to hypovolemia and inadequate kidney perfusion. Patients often undergo “ping-pong” volemia in hospitalized settings, a situation where hypovolemia is over-treated with fluid, followed by an over-treatment of diuretics that leads to new hypovolemia, followed by new fluid treatment.

This case also illustrates an important surgical complication. Surgery for replacement of part of the aorta may inadvertently or intentionally sacrifice branches that supply the intestines. In many patients, adequate collateral circulation is available. However, the outcome is difficult to predict; if the collateral circulation of the intestines is not sufficient, than death of parts or all of the intestines may occur.

In this example, a 58-year-old man with a history of diabetes mellitus, hypertension and congestive heart failure due to hypertensive cardiomyopathy comes to the operating room for elective resection of an abdominal aortic aneurysm and placement of an aortic to bilateral iliac graft. The surgery is fairly uneventful although lasting about 6 hours. During the surgery, the patient's urine output is lower than the normal range but his vital signs otherwise appear stable.

A tissue state monitor is placed in the upper GI tract. It is assumed that the reading of the monitor is completely reliable and precise indicator of both kidney and GI micro-perfusion. The output of the monitor is from 0-100. A measure of 100 represents fully healthy organs receiving excess oxygen and glucose. Measures of energy between 70 and 99 represent organs receiving enough oxygen and glucose to perform tissue function (organ compromise) with 70 representing the pre-dysoxia point for that tissue. Measures of energy between 50 and 69 represent organs receiving enough oxygen and glucose to survive indefinitely but not perform their tissue function (organ failure). Measures of energy between 20 and 49 represent organs not receiving enough oxygen and glucose to survive indefinitely. Measures of energy below 20 represent dead or imminently dying organs.

The monitor initially reads 100, suggesting that despite the low urine output; the patient is receiving perfusion to his abdominal organs. Thus, with assurance that end organ perfusion is adequate, the most likely cause of the patient's poor urine output is the release of stress hormones. If this is the case, there is no reason to take action at this time. The addition of fluid to treat the low urine output will result in fluid overload. The use of diuretics to stimulate urine output will result in fluid depletion.

### EXAMPLE 13

This example has the same facts as Example 12. In addition to the stated patient situation, the tissue state monitor initially reads 75, suggesting that perfusion to the patient's abdominal organs is considerably compromised. A clinical decision is made to administer fluids. The tissue state monitor begins to climb as fluids are taken on. The tissue state monitor now reads 100, even though the urine output is still very low. There may be reasons to continue administering fluid at this point. The patient's vital signs are stable and tissue perfusion has returned to normal. Some additional fluid may be warranted at this point to offer a hydration buffer, but continuing high-volume fluid puts the patient in danger of fluid overload.

### EXAMPLE 14

This example has the same facts as Example 12. In addition to the stated patient situation, the monitor initially reads 100, suggesting that despite the low urine output; the patient is receiving perfusion to his abdominal organs. Near the end of the surgery, the tissue state monitor precipitously falls to 55, indicating that tissue energy has fallen below that of productive organ function.

The patient is developing surgical end organ failure. This is a serious condition that needs closer scrutiny by both the surgeon and the anesthesiologist in order to avoid a morbid or mortal outcome. With all major surgical procedures, this patient runs the risk of surgical shock. In addition, given the nature of the surgery, this patient is at risk for ischemic bowel secondary to his vascular operation. The surgeon now has the opportunity to evaluate the bowel more closely to determine if the procedure warrants further remodeling or a prophylactic partial bowel resection. Before transfer to the ICU, the anesthesiologist has the opportunity to optimize the fluid, oxygenation, and cardiovascular status of the patient for adequate tissue perfusion.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A patient tissue state monitoring system comprising:
a light source;
a light detector;
a probe adapted to be inserted into a patient to transmit light from the light source to an organ tissue site and to direct light from the organ tissue site to the detector; and a processor programmed to determine tissue state with respect to a tissue site pre-dysoxia point from a fluorescence emission detected by the detector and to provide an indication of tissue state through an output device.

2. The patient tissue state monitoring system of claim 1 further comprising a light conduit extending distally from the light source to the probe.

3. The patient tissue state monitoring system of claim 1 further comprising a light conduit extending proximally from the probe to the light detector.

4. The patient tissue state monitoring system of claim 1 wherein the processor is further programmed to determine tissue state with respect to a tissue site dysoxic point from a fluorescence emission detected by the detector.

5. The patient tissue state monitoring system of claim 1 wherein the processor is further programmed to determine tissue state with respect to a tissue site oxygenation critical point from a fluorescence emission detected by the detector.

6. The patient tissue state monitoring system of claim 1 wherein the indication comprises a numerical value corresponding to the fluorescence emission.

7. The patient tissue state monitoring system of claim 1 wherein the processor is programmed to determine tissue NADH concentration.

8. A method of monitoring a patient tissue state comprising:
monitoring an aerobic energy production level of an organ tissue site;
determining tissue state with respect to a tissue site pre-dysoxia point from the monitored aerobic energy production level; and
providing an output of the tissue state.

9. The method of claim 8 wherein the monitoring step comprises monitoring fluorescence emission of the organ tissue site.

10. The method of claim 8 wherein the organ tissue site is in a gastro-intestinal tract.

11. The method of claim 8 wherein the organ tissue site is in a bladder.

12. The method of claim 8 wherein the organ tissue site is in a urethra.

13. The method of claim 8 wherein the determining step comprises determining tissue NADH concentration.

14. The method of claim 8 wherein providing an output comprises visually reporting a numerical value corresponding to the fluorescence emission.

15. The method of claim 8 determining tissue state with respect to a tissue site dysoxic point from the monitored aerobic energy production level.

16. The method of claim 8 determining tissue state with respect to a tissue site oxygenation critical point from the monitored aerobic energy production level.

17. A method of warning of a likely pre-dysoxia point of a kidney comprising:
monitoring an aerobic energy production level of a stomach tissue site;
determining a stomach tissue site tissue state with respect to a tissue pre-dysoxia point from the monitored aerobic energy production level; and
providing an output that the stomach of the tissue state.

18. The method of claim 17 wherein the monitoring step comprises monitoring fluorescence emission of the stomach tissue site.

19. The method of claim 17 wherein the determining step comprises determining tissue NADH concentration.

20. The method of claim 17 wherein providing an output comprises visually reporting a numerical value corresponding to the fluorescence emission.
